(19) United States
(12) Patent Application Publication
(10) Pub. No.: US 2003/0157081 A1
(43) Pub. Date: Aug. 21, 2003

(54) Methods and compositions for suppression of neurodegeneration

(76) Inventors: Nancy M. Bonini, Narberth, PA (US); Henry L. Paulson, Iowa City, IA (US)

Correspondence Address: Licata & Tyrrell P.C., 66 E. Main Street, Marlton, NJ, 08053, US

(21) Appl. No.: 10/283,409

(22) Filed: Oct. 28, 2002

Related U.S. Application Data
(60) Provisional application No. 60/339,598, filed on Oct. 26, 2001.

(51) Int. Cl. A61K 38/43

(52) U.S. Cl. 424/94.1

(57) ABSTRACT

Methods for changing solubility of expanded polyglutamine protein, suppressing polyglutamine toxicity, and treating neurodegenrative disorder by administering to the subject a Hsp40 chaperone or an agent which increases expression or activity of a Hsp40 chaperone, preferably in combination with a Hsp70 chaperone or an agent which increases expression or activity of a Hsp70 chaperone are provided. Also provided are compositions for treating neurodegenrative disorders containing a Hsp40 chaperone or an agent which increases activity or expression of a Hsp40 chaperone and a Hsp70 chaperone or an agent which increases activity or expression of a Hsp70 chaperone as well as methods for identifying such agents.

### INTRODUCTION

This application claims the benefit of priority of U.S. Provisional Application Ser. No. 60/339,598, filed Oct. 26, 2001, which is herein incorporated by reference in its entirety.

### FIELD OF THE INVENTION

The present invention provides compositions and methods for changing solubility of expanded polyglutamine protein and suppressing polyglutamine toxicity in a subject via administration of a Hsp40 chaperone or an agent which increases expression and/or activity of a Hsp40 chaperone alone, or more preferably, in combination with a Hsp70 chaperone or an agent which increases expression and/or activity of a Hsp70 chaperone. The compositions and methods of the present invention are useful in treating neurodegenerative disorders associated with abnormal protein aggregation. Also provided are methods for identifying agents useful in treating neurodegenerative disorders associated with abnormal protein aggregation which comprise determining a test agent's ability to increase expression and/or activity of a Hsp40 or Hsp70 chaperone.

### BACKGROUND OF THE INVENTION

At least eight human neurodegenerative diseases, including Huntington's disease, share a common molecular mechanism involving expansion of a polyglutamine tract within the respective disease proteins (Lin et al. Neuron 1999 24:499-502; Perutz, M. TIBS 1999 24:58-63; Zoghbi, H. Y. and Orr, H. T. Mol. Cell. Biol. 2000 19:7751-7758). Expanded polyglutamine confers dominant toxicity on the otherwise unrelated disease proteins, leading to neuronal dysfunction and cell loss. The expanded polyglutamine proteins form nuclear inclusions that are associated with chaperones, thus indicating misfolding of the protein.

In human disease tissue, transgenic animal models, and transfected cells, expanded polyglutamine protein has been shown to undergo intracellular aggregation, in most cases forming nuclear inclusions (Lin et al. Neuron 1999 24:499-502; Perutz, M. TIBS 1999 24:58-63; Zoghbi, H. Y. and Orr, H. T. Mol. Cell. Biol. 2000 19:7751-7758). These aggregates are immunoreactive for ubiquitin, various molecular chaperones and components of the proteasome complex (Chai et al. Hum. Mol. Gen. 1998 8:673-682; Cummings et al. Nature Genetics 1998 19:148-154; Chai et al. J. Neurosci. 1999 19:10338-10347; Stenoien et al. Hum. Mol. Genet. 1999 8:731-741). This is indicative of the aggregated disease protein being recognized by cells as abnormal, and that chaperone and proteasome recruitment to inclusions may serve to refold, disaggregate and/or degrade the mutant protein. Consistent with this view, overexpression of select molecular chaperones in mammalian cells in culture has been shown to reduce aggregate formation by several polyglutamine disease proteins (Cummings et al. Nature Genetics 1998 19:148-154; Chai et al. J. Neurosci. 1999 19:10338-10347; Stenoien et al. Hum. Mol. Genet. 1999 8:731-741).

To elucidate molecular mechanisms underlying disease, polyglutamine toxicity has been modeled in Drosophila melanogaster through targeted expression of polyglutamine disease proteins (Jackson et al. Neuron 1998 21:633-642; Warrick et al. Cell 1998 93:939-949; Kazemi-Esfarjani, P. and Benzer, S. Science 2000 287:1837-1840); Marsh et al. Human Mol. Genet. 2000 9:13-25). Expression of expanded polyglutamine protein in Drosophila induces late-onset, progressive neurodegeneration, indicating that mechanisms of polyglutamine-induced neural degeneration are conserved in flies. Drosophila thus provide a genetic system in which to address modulation of polyglutamine neurotoxicity in vivo.

Coexpression of the heat shock protein chaperone Hsp70 with toxic polyglutamine protein suppresses neurodegeneration in flies (Warrick et al. Nat. Genet. 1999 23:425-428). Increasing Hsp70 activity has also been shown to have protective effects on neural toxicity in vertebrates (Plumier et al. Cell Stress & Chaperones 1997 2:162-167).

Two J-domain-containing proteins have also been shown to ameliorate toxicity of an isolated polyglutamine domain in flies (Kazemi-Esfarjani, P. and Benzer, S. Science 2000 287:1837-1840).

It has now been found that increasing expression of a heat shock protein Hsp40 chaperone and in particular a Hdj1 class of chaperones also provides protection against neural toxicity. Further, as demonstrated herein, Hsp40 and Hsp70 chaperones act synergistically to provide increased protection against neural toxicity.

### SUMMARY OF THE INVENTION

An object of the present invention is to provide methods for changing solubility of expanded polyglutamine protein in a subject comprising administering to the subject a Hsp40 chaperone or an agent which increases expression and/or activity of a Hsp40 chaperone alone or, more preferably, in combination with a Hsp70 chaperone or an agent which increases expression and/or activity of a Hsp70 chaperone.

Another object of the present invention is to provide methods for suppressing polyglutamine toxicity in a subject comprising administering to the subject a Hsp40 chaperone or an agent which increases expression and/or activity of a Hsp40 chaperone alone, or more preferably, in combination with a Hsp70 chaperone or an agent which increases expression and/or activity of a Hsp70 chaperone.

Another object of the present invention is to provide methods for treating a subject suffering from a neurodegenerative disorder associated with abnormal protein aggregation comprising administering to the subject a Hsp40 chaperone or an agent which increases expression and/or activity of a Hsp40 chaperone alone or, more preferably, in combination with a Hsp70 chaperone or an agent which increases expression and/or activity of a Hsp70 chaperone.

Another object of the present invention is to provide methods for identifying agents useful in treating neurodegenerative disorders associated with abnormal protein aggregation comprising determining a test agent's or Hsp70 chaperone. In these methods, the ability of the test agent to increase expression and/or activity of the Hsp40 or Hsp70 chaperone is indicative of the test agent being useful in treating neurodegenerative disorders associated with abnormal protein aggregation.

Yet another object of the present invention is to provide compositions useful for treatment of a neurodegenerative disorder associated with abnormal protein aggregation. Such compositions comprise a Hsp40 chaperone or an agent which increases activity and/or expression of a Hsp40 chaperone and a Hsp70 chaperone or an agent which increases activity and/or expression of a Hsp70 chaperone.

### DETAILED DESCRIPTION OF THE INVENTION

Many human neurodegenerative diseases, including but not limited to, Alzheimer's disease and Parkinson's disease, are associated with abnormal protein aggregation. In these diseases, as in polyglutamine diseases, the abnormal aggregates are often ubiquitinated, suggesting involvement of protein folding and degradation pathways in the disease process (Kakizuka, A. Trends Genet. 1998 14:396-402). Thus, there is believed to be a common pathogenic mechanism underlying these different late-onset, progressive neurological conditions.

The present invention provides methods and compositions for suppressing neural toxicity in neurodegenerative disorders by increasing expression and/or activity of the heat shock protein Hsp40 alone, or in combination with increasing the expression and/or activity of the heat shock protein Hsp70. As shown herein, compositions which increase the expression and/or activity of Hsp40 alone, or more preferably in combination with an increase in the expression and/or activity of Hsp70, increase the solubility of expanded polyglutamine protein and are expected to be useful in the treatment of polyglutamine and other human neurodegenerative diseases associated with abnormal protein aggregation.

Using a Drosophila model of polyglutamine disease, it has now been demonstrated that Hsp40 molecular chaperones show substrate specificity for polyglutamine protein, and that Hsp40 and Hsp70 synergize in suppression of neurodegeneration. Western analysis revealed that, although nuclear inclusions are still present, these chaperones increased solubility of expanded polyglutamine protein. Accordingly, the present invention provides methods and compositions for suppressing the toxicity associated with altered biochemical properties of expanded polyglutamine protein via Hsp40 or an agent which increases activity and/or expression of Hsp40 alone, or in combination with Hsp70 or an agent which increases activity and/or expression of Hsp70.

The role of heat shock protein (Hsp) chaperones in polyglutamine disease was examined in Drosophila using a disease protein for one of the most common polyglutamine diseases, spinocerebellar ataxia type 3, also known as Machado-Joseph disease (SCA3/N4JD; Kawaguchi et al. Nature Genetics 1994 8:221-228). Normally, the SCA3 protein ataxin-3 has a polyglutamine domain of 12-40 glutamine residues. In a disease state, however, this domain becomes expanded to 60-80 residues. In the fly, expression of a truncated form of ataxin-3 containing expanded polyglutamine induces progressive neurodegeneration accompanied by the formation of nuclear inclusions, to which endogenous Hsp70, the major stress-induced molecular chaperone in Drosophila, localizes (Warrick et al. Cell 1998 93:939-949; Warrick et al. Nat. Genet. 1999 23:425-428). Hsp70 activity is modulated by Hsp40 molecular chaperones. Accordingly, the role and specificity of Hsp40 chaperones in modulation of polyglutamine disease in Drosophila was examined.

Hsp40 chaperones are also known as DNAJ proteins after the bacterial homologue. Hsp40 proteins contain a J-domain that is important for interaction with Hsp70, and a C-terminal domain that is critical for interaction with substrate proteins (Bukau, B. and Horwich, A. Cell 1998 92:351-366). The different C-terminal domains of the Hsp40 proteins have been suggested to impart substrate specificity to particular Hsp40 chaperones (Kelly, W. L. Biochem. Sci. 1998 23:222-227).

In these experiments, the effects of increasing Hsp40 expression at mitigating polyglutamine-induced degeneration were first examined. For these experiments, transgenic flies expressing a cDNA encoding dHdj1, the fly orthologue of the human Hsp40 chaperone family member Hdj1, were produced. Expression was achieved with the GAL4/UAS system (Brand, A. H. and Perrimon, N. Development 1993 118:401-415). A cDNA encoding fly dHdj1 was subcloned into the PUAST transformation vector and transgenic flies were generated.

Expression was directed to the eye with gmr-GAL4, a promoter line that directs expression to all cells of the eye, including accessory pigment cells and photoreceptor neurons. Expression of truncated ataxin-3 with expanded polyglutamine (MJDtr-Q78) in the eye induces massive degeneration with loss of external pigmentation and internal retinal structure (Warrick et al. Cell 1998 93:939-949). Upon coexpression of dHdj1 with the MJDtr-Q78 protein, the external eye structure was restored. Internal eye structure, which normally undergoes massive degeneration upon expression of the mutant protein, was also restored by dHdj1 coexpression, with the eye showing preservation of photoreceptor rhabdomere specializations.

Several types of control experiments were performed to confirm that suppression was specific to dHdj1 expression. First, it was previously shown that simply coexpressing two proteins by the same GAL4 promoter line has no effect on neurodegeneration (Warrick et al. Nat. Genet. 1999 23:425-428). Second, dHdj1 did not modulate gene expression by the GAL4/UAS system. For example, coexpression of dHdj1 with other proteins that induce a phenotype, such as the cell death protein Hid, did not modify the reduced eye phenotype caused by Hid expression. Coexpression with Hid also indicated that dHdj1 does not function by preventing programmed cell death. Moreover, immunoblot analysis showed that coexpression of dHdj1 did not decrease levels of polyglutamine protein expression. Taken together, these data indicate that dHdj1, like Hsp70, selectively mitigates polyglutamine protein toxicity.

Further analysis revealed that dHdj1 also ameliorated progressive degeneration induced by polyglutamine protein. Flies expressing the mutant disease protein less strongly in the eye, such that a more intact eye structure is present upon emergence of the adult were used to examine progressive loss of neurons over time. As these flies mature, they display dramatic retinal degeneration. Coexpression of dHdj1 dramatically restored structure of the eye at emergence of the adult, as well as mitigating progressive neural loss over time.

To determine whether dHdj1 could suppress polyglutamine toxicity throughout the nervous system, expression of mutant polyglutamine protein was directed to all neurons with elav-GAL4. Normally, strong expression of expanded polyglutamine protein by elav-GAL4 is lethal, with no adult flies emerging (Warrick et al. Cell 1998 93:939-949). However, coexpression of dHdj1 suppressed lethality such that adult flies emerged and lived as long as two weeks.

In vitro in transfected cells, Hsp40 chaperones have been shown to decrease aggregation of expanded polyglutamine protein (Cummings et al. Nature Genetics 1998 19:148-154; Chai et al. J. Neurosci. 1999 19:10338-10347; Stenoien et al. Hum. Mol. Genet. 1999 8:731-741). In contrast, in in vivo fly experiments, the mutant disease protein still formed prominent nuclear inclusions in the presence of dHdj1, indicating that the mechanism of dHdj1 suppression in vivo does not involve elimination of nuclear inclusions. Further, immunostaining revealed that dHdj1 was associated with nuclear inclusions, thus indicating that exogenous dHdj1 colocalized with aggregated disease protein in vivo.

Hsp40 proteins have two critical functional domains. The first is the J-domain which interacts with and stimulates the ATPase activity of Hsp70 (Bukau, B. and Horwich, A. Cell 1998 92:351-366). The second is the C-terminal domain which is important for interactions with substrate proteins. Mutants constructs of dHdj1 were prepared to determine which of these domains is important for suppression of neurodegeneration. In the first construct (dHdj1ΔJ), the J-domain was deleted, which is predicted to eliminate interaction with Hsp70 in vivo. In a second construct (dHdj1.G295D), a point mutation was made within the C-terminal region that is predicted to interfere with substrate binding (Lu, Z. and Cyr, D. M J. Biol. Chem. 1998 273:5970-5978). External eye structure remained normal when either mutant dHdj1 protein was expressed on its own, indicating that they were not toxic transgenes. However, when coexpressed with expanded polyglutamine protein, both mutant dHdj1 proteins not only failed to suppress, but instead enhanced the disease phenotype. Weak expression of MJDtr-Q78 normally results in adult flies with only a mildly disrupted eyes (Warrick et al. Cell 1998 93:939-949). However, upon coexpression with dHdj1ΔJ or dHdj1.G295D, the eye displayed more severe degeneration, seen by the enhanced loss of pigmentation and collapse of the eye. These studies indicate that both the Hsp70 interaction and the substrate binding domains are important for dHdj1-mediated suppression of polyglutamine-induced degeneration.

Further, the enhanced toxicity observed with these mutants is indicative of endogenous levels of Hsp40 and Hsp70 chaperones being required to modulate toxicity of polyglutamine protein in vivo. Normal cellular levels of Hsp40 and Hsp70, or levels induced as part of a stress response to the mutant protein, are believed to aid in combating deleterious effects of the protein, and contribute to the typically late onset nature of disease in humans. These experiments with mutant Hsp40 proteins indicate that augmenting levels of Hsp40 and Hsp70 induced as part of a stress response in vivo may also be effective in counteracting deleterious actions of the toxic protein.

The presence of Hsp40 proteins with divergent C-terminal domains has been suggested to underlie differential substrate specificity of various Hsp40 chaperones (Kelly, W. L. Trends Biochem. Sci. 1998 23:222-227). For example, divergent C-terminal domains have been linked to different functional aspects of the yeast Hsp40 proteins Sis I and Ydj I (Lu, Z. and Cyr, D. M. J. Biol. Chem. 1998 273:27824; Yan, W. and Craig, E. A. Mol. Cell. Biol. 1999 19:7751-7758). Phylogenetic analysis suggests three groupings of J-domain proteins: two classes defined by Hdj1 and Hdj2, which are the major Hsp40 chaperones involved in protein folding, and a third class of J-domain containing proteins of diverse functions. A genetic screen in Drosophila identified two J-domain containing proteins as suppressors of polyglutamine toxicity, dHdj1 and dTPR2 (tetratricopeptide repeat protein 2), although the effectiveness of the proteins, the protein domains important to activity, and the synergy with other heat shock protein chaperones were not addressed (Kazemi-Esfarjani, P. and Benzer, S. Science 2000 287:1837-1840).

Experiments were performed to examine in vivo specificity of selected Hsp40 chaperones to modulate polyglutamine toxicity. It was found that a second Hsp40 chaperone, dHdj2, was less effective at suppression than the Hsp40 chaperone dHdj1. dHdj1 and dHdj2 share a J-domain, but have divergent C-terminal domains, potentially reflecting different substrate specificities. To address the specificity of dHdj1 suppression, transgenic flies were made that express dHdj2, the fly homologue of human Hdj2. Hdj2 has been shown to decrease aggregate formation of several polyglutamine proteins, including ataxin-3, in transfected cells (Cummings et al. Nature Genetics 1998 19:148-154; Chai et al. J. Neurosci. 1999 19:10338-10347; Stenoien et al. Hum. Mol. Genet. 1999 8:731-741). Accordingly, several dHdj2 transgenic lines that express dHdj2 at levels one- to two-fold that of the strongest dHdj1 transgenic line made were also produced. Coexpression of any of these dHdj2 transgenic lines with polyglutamine protein resulted in weak suppression of neurodegeneration when compared to dHdj1 suppression. These results are indicative of selective interactions of specific Hsp40 chaperones with expanded polyglutamine protein in vivo. Further, these experiments are indicative of the Hdj1 class of chaperones being preferred chaperones for use in modulating the toxicity of the expanded ataxin-3 protein as well as other aggregated proteins.

The functional interactions between dHdj1 and Hsp70 in mediating suppression of polyglutamine disease were also examined. When expanded polyglutamine protein is expressed strongly in the eye, coexpression of either Hsp70 or dHdj1 resulted in complete suppression of the external eye degeneration, but only partial suppression of the internal eye structure. However, when dHdj1 and Hsp70 were expressed simultaneously with the disease protein, suppression was more complete with the rescued eye now showing specializations of the photoreceptor neurons. This indicates that coexpression of both chaperones was more effective than expression of either alone. Similar results were found when expression was directed to all neurons with elav-GAL4. Normally, no adult flies emerge when the mutant disease protein is expressed strongly throughout the nervous system. Coexpression of dHdj1 or Hsp70 alone resulted in partial suppression of the early death phenotype, with rescued female flies surviving up to about 2 weeks. Although coexpression of both dHdj1 and Hsp70 did not lead to a significant extension in lifespan beyond that of each chaperone when expressed individually, there was a striking effect on male survival. Males express twice the level of mutant disease protein because the elav-GAL4 insertion is on the X chromosome, and thus is subject to dosage compensation. Expression of either dHdj1 or Hsp70 alone resulted in some adult male survival relative to females. Up to about 20% was observed with dHdj1. However, upon coexpression of both chaperones, approximately equal numbers of male and female flies survived with expanded polyglutamine protein.

To further define the nature of the interaction between dHdj1 and Hsp70, recombinant fly lines that contained weakly-expressing transgenic insertions of both dHdj1 and Hsp70 were generated. If the two proteins interacted in a non-linear manner to suppress polyglutamine toxicity, strong suppression when low levels of both chaperones were expressed together was anticipated. However, an interaction between dHdj1 and Hsp70 was not detected by this approach.

This raised the possibility that the increased suppression observed when both chaperones are expressed might be a simple dosage effect, such that two doses of chaperone activity are more effective than one. However, control experiments revealed that expression of two doses of dHdj1 or Hsp70 alone in the absence of expanded polyglutamine protein were deleterious to eye morphology. The deleterious effects included loss of pigmentation upon two doses of dHdj1 chaperone and, upon one dose, altered nuclear position of pigment cells; for Hsp70, two doses causes severe rough eye phenotype, whereas one dose had similar (but less severe) alteration of nuclear position of pigment cells. However, when one copy each of dHdj1 and Hsp70 were coexpressed, eye morphology was restored toward normal . Thus, these results indicate that dHdj1 and Hsp70 have differing, albeit linked, biological activities in vivo. Similarly, although expression of dHdj1 or Hsp70 suppressed polyglutamine toxicity when expressed in the nervous system with elav-GAL4, expression of dHdj1 alone resulted in shortened lifespan that was partially suppressed upon coexpression of dHdj1 with Hsp70.

Thus, these experiments are supportive of a synergistic interaction between dHdj1 and Hsp70 in suppressing polyglutamine toxicity. Although either dHdj1 or Hsp70 separately had a significant ability to restore eye structure in the presence of expanded polyglutamine protein, simultaneous expression of both chaperones resulted in an even better internal eye structure, with less progressive degeneration over time in the adult. Moreover, there was synergy in suppression of pre-adult lethality in males.

Interestingly, deleterious effects were observed upon overexpression of either chaperone alone, especially dHdj1, that were minimized upon coexpression of the two proteins together. Hsp40 and Hsp70 proteins commonly function as pairs in protein folding, however, and the deleterious effects of expressing either chaperone alone may be due to selective augmentation of the levels of one chaperone in the absence of sufficient levels of the other. These studies are indicative of a potentially safer approach to treatment of these neurodegenerative diseases via administration of a composition which comprises Hsp40 and Hsp70 or agents which increase activity and/or expression of Hsp40 and Hsp70 or via up-regulation of the stress response at the level of the transcriptional activator heat shock factors, which would achieve the physiological balance of stress factors required to combat the toxicity of the protein.

To determine the mechanism of suppression by dHdj1 and Hsp70 nuclear inclusion formation was examined in developing eye tissue and adult brain sections. As before, no change in nuclear inclusion formation by light microscopic analysis was observed, either during their formation or in the adult. However, upon extension of these studies to examine protein aggregation at the biochemical level by immunoblot analysis, a striking change in solubility of the expanded polyglutamine protein upon coexpression with molecular chaperones was observed.

Normally, when expanded polyglutamine protein is extracted from fly heads, the protein runs as an SDS-insoluble complex within the stacking gel, with very little protein visible as a monomer. However, upon coexpression of dHdj1 and Hsp70, significant levels of monomeric protein were detectable. The normal lack of monomeric protein was not due to loss of eye tissue, because the result was the same whether the polyglutamine protein was expressed weakly (when no retinal degeneration has yet occurred in adult flies) or strongly (when a significantly degenerate morphology is observed).

To further correlate monomer expression with suppression of polyglutamine toxicity, monomer levels observed upon expression of dHdj1 were compared with monomer levels observed upon expression of dHdj2. In these experiments, similar levels of an Hsp40 chaperone were expressed. However, strong suppression of toxicity was observed with expression of dHdj1 while weak suppression was seen with expression of dHdj2. Consistent with the amount of monomer reflecting the ability of the chaperone to modulate toxicity of the protein, more monomer was observed upon dHdj1 coexpression when toxicity was greatly suppressed, than upon dHdj2 coexpression when toxicity was modestly suppressed. These observations are indicative of modulation of protein solubility being important in disease suppression.

Thus, in one embodiment, the present invention provides methods for changing, or more preferably increasing, solubility of expanded polyglutamine protein in a subject by administering to the subject a Hsp40 chaperone or an agent which increases expression and/or activity of a Hsp40 chaperone. In a preferred embodiment, the Hsp40 chaperone is from the Hdj1 class of chaperones. In this method it is also preferred that the subject be administered a Hsp70 chaperone or an agent which increases expression and/or activity of a Hsp70 chaperone. When used in combination, it is believed that these chaperones act synergistically to alter the structure, and hence solubility, of the disease protein.

As also shown herein, administration of Hsp40 and more preferably, Hsp40 in combination with Hsp70 suppressed polyglutamine toxicity. Thus, another aspect of the present invention relates to a method for suppressing polyglutamine toxicity in a subject by administering to the subject a Hsp40 chaperone or an agent which increases expression and/or activity of a Hsp40 chaperone. In this method it is also preferred that the Hsp40 chaperone be from the Hdj1 class of chaperones. Further, it is preferred that the subject be administered a Hsp70 chaperone or an agent which increases expression and/or activity of a Hsp70 chaperone as well.

The ability of a Hsp40 chaperone, or more preferably a Hsp chaperone in combination with a Hsp70 chaperone to increase disease protein solubility and decrease polyglutamine toxicity in vivo is indicative of this therapy being useful in the treatment of neurodegenerative disorders associated with abnormal protein aggregation. Accordingly, the present invention also provides methods for treatment of a subject suffering from a neurodegenerative disorder associated with abnormal protein aggregation which comprises administering to the subject a Hsp40 chaperone or an agent which increases expression and/or activity of a Hsp40 chaperone alone, or more preferably in combination with a Hsp70 chaperone or an agent which increases expression and/or activity of a Hsp70 chaperone. In these methods it is preferred that the Hsp40 chaperone be from the Hdj1 class of chaperones. Examples of neurodegenerative disorders associated with abnormal protein aggregation which can be treated by the methods of the present invention include, but are in no way limited to, Alzheimer's disease, Parkinson's disease, Huntington's disease and polyglutamine diseases.

In the methods of the present invention, the subject may be administered the heat shock protein itself. For Hsp40 it is preferred that a Hdj1 class of chaperones be used. Alternatively, the subject may be administered an agent which increases the activity and/or levels of the heat shock protein. Examples of such agents include, but are not limited to, peptidomimetics of the heat shock proteins and gene therapy agents expressing the heat shock proteins. In a preferred embodiment, the agent up-regulates the stress response in vivo at the level of the transcriptional activators of heat shock factors so that a physiological balance of Hsp40 and Hsp70 required to combat the toxicity of the protein is achieved.

Agents for use in treating neurodegenerative disorders associated with abnormal protein aggregation in accordance with the methods of the present invention can be identified routinely be those of skill in the art upon reading this disclosure by assessing the ability of a test agent to increase expression and/or activity of a Hsp40 or Hsp70 chaperone. Test agents demonstrated to increase expression and/or activity of a Hsp40 or Hsp70 chaperone are expected to be useful in treating neurodegenerative disorders associated with abnormal protein aggregation. For Hsp40 preferred agents are those which increase activity and/or expression of a Hdj1 class of chaperones.

In the methods of the present invention, the subject may be administered a Hsp40 or an agent which increases the activity and/or expression of Hsp40 alone or, more preferably, in combination with Hsp70 or an agent which increases the activity and/or expression of Hsp70. By “in combination” it is meant to include simultaneous administration of each heat shock protein or agent as well as sequential administration of a heat shock protein or agent. When administered simultaneously, it is preferred that the heat shock proteins or agents be administered as a single composition.

Accordingly, another aspect of the present invention relates to compositions for treatment of a neurodegenerative disorder associated with abnormal protein aggregation which comprises a Hsp40 chaperone or an agent which increases activity and/or expression of a Hsp40 chaperone and a Hsp70 chaperone or an agent which increases activity and/or expression of a Hsp70 chaperone. For these compositions it is preferred that the Hsp40 chaperone be from the Hdj1 class of chaperones. Also preferred for the compositions is at least one of the agents augmenting levels of Hsp40 and Hsp70 induced as part of a stress response in the subject.

The formulation of compositions of the present invention and their subsequent administration is believed to be within the skill of those in the art. Dosing is dependent on severity and responsiveness of the disease state to be treated, with the course of treatment lasting from several days to several months, or until a cure or alleviation of the disease state is achieved. Optimal dosing schedules can be calculated from measurements of drug accumulation in the body of the subject. Optimum dosages, dosing methodologies and repetition rates can also be routinely determined by those of skill in the art. Optimum dosages may vary depending on the relative potency of agents, and can generally be estimated based on EC₅₀s found to be effective in in vitro and in vivo animal models. Repetition rates for dosing can also be determined routinely based on measured residence times and concentrations of the composition in bodily fluids or tissues. Following successful treatment, it may be desirable to have the patient undergo maintenance therapy to prevent the recurrence of the disease state, wherein the composition is administered in lower maintenance doses with less frequency.

The following nonlimiting examples are provided to further illustrate the present invention.

### EXAMPLES

### Example 1

Drosophila Strains

Flies were grown on standard corn meal molasses media supplemented with dry yeast, at 25° C. Fly lines bearing UAS-MJDtr-Q27, UAS-MJDtr-Q78 and hsp70 (UAS-HspA1L) have been described (Warrick et al. Cell 1998 93:939-949; Warrick et al. Nat. Genet. 1999 23:425-428). For lines expressing dHdj1 and dHdj2, cDNA clones encoding these proteins were obtained from Research Genetics (Huntsville, AL), a FLAG tag was added to the C-terminus of the protein by PCR, the constructs were sequenced and subcloned into the PUAST transformation vector (Brand, A. H. and Perrimon, N. Development 1993 118:401-415). For dHdj1, primers used were: N-terminal, 5′-CGGAATTCGTCGACATGGGCAAAGA CTTCTACAAGATTCTGGGCC-3′ (SEQ ID NO:1), and C-terminal 5′-GGTCTAGACTACTTGTCGTCGTCGTCATCCTTGTAATCGTTGGGCAGCAGCTCCGA CAGCTGATTCTGC-3′ (SEQ ID NO:2). For dHdj2, primers used were: N-terminal, 5′-CGCTCGAGGTCGACATGGACAACCTAAATTTATACGACGTTCTTAAAGTGGC-3′ (SEQ ID NO:3), and C-terminal, 5′-GGTCTAGACTACTTGTCGTCGTCGTCATCCTTGTAATCAGCCGTCTGGCACTGTAC GCCCTCAAAGTGAGACG-3′ (SEQ ID NO:4). The J-domain deletion mutation in dHdj1 was made using a PCR primer to delete the N-terminus of the protein (5′-CGGAATTCGTCGACATGGGACCAGATGGCGGCGGTCAGCCG-3′ (SEQ ID NO:5)). The G295D mutation was made using primers to the sequence 5′-GGATCAACGGACTAGATCTGCCGGTGCCC-3′ (SEQ ID NO:6) and the QuikChange site-directed mutagenesis kit (Stratagene, La Jolla, Calif.). The constructs bearing mutant proteins were sequenced and subcloned into the pUAST vector. Transgenic lines were generated in the w¹¹⁸ background (Rubin, G.M. and Spradling, A.C. Science 1982 218:348-353). Fly lines bearing gmr-GAL4 and elav-GAL4 (line C155) were obtained from the Drosophila Bloomington Stock Center; gmr-hid was obtained from the Department of Biology, M.I.T.; gmr-P35 lines were obtained from Department of Biology, Caltech).

### Example 2

Microscopy, Immunohistochemistry and Immunoblot Analysis

Eye discs were dissected and stained with antibodies as described (Warrick et al. Cell 1998 93:939-949). HA-tagged polyglutamine protein was detected with a rabbit polyclonal antibody to hemagglutinin (Y-11, 1:200, Santa Cruz Biotechnology, Santa Cruz, Calif.). Drosophila Hsp70 protein was detected with a monoclonal antibody to the fly protein (MAB-007, 1:200, Affinity BioReagents, Golden, Co.). Human Hsp70 was detected with an antibody specific to the human protein (SPA812, 1:200, StressGen, Victoria, Canada). FLAG-labeled proteins were detected using anti-FLAG M2 (1:100; Sigma, St. Louis, Mo.). Secondary antibodies were conjugated to fluorescein (1:500) or Texas red (1:50, Jackson ImmunoResearch, West Grove, Pa.). For staining of frozen sections, adult heads (0-1 day old) were embedded in OCT, 12 μm serial sections were cut and collected on subbed slides. Tissue was fixed in 4% paraformaldehyde, then stained with appropriate antibodies and DAPI (0.5 μg/ml for 5 minutes). For plastic sections, adult heads (0-1 day old) were fixed in paraformaldehyde and embedded in epon for 1 μm horizontal sections, then stained with toluidine blue and methylene blue as described (Warrick et al. Cell 1998 93:939-949). Confocal microscopy was performed on a Leica model TCS SP ultraviolet and visible confocal imaging spectrophotometer microscope. For light microscopy, tissue was viewed on a Leica DRMB compound fluorescence microscope or a Leica MZ12 stereomicroscope, equipped with a Leica DC200 digital camera.

### Example 3

Immunoblot Analysis

Adult fly heads of the appropriate genotypes were prepared and analyzed by SDS-PAGE and immunoblot as previously described (Warrick et al. Nat. Genet. 1999 23:425-428). Gels were 12.5% polyacrylamide, with a 4% stacking gel. Antibodies used were anti-HA (12CA5, 1:3000; Boehringer Mannheim, Indianapolis, Ind.), anti-FLAG M2 (1:440), anti-H-Hsp70 (1:10,000), and anti-β-tubulin (E7, 1:1000; Developmental Studies Hybridoma Bank). Secondary antibodies were goat anti-mouse and goat anti-rabbit (1:4000, Boehringer Mannheim, Indianapolis, Ind.).

What is claimed is:

1. A method for changing solubility of expanded polyglutamine protein in a subject comprising administering to the subject a Hsp40 chaperone or an agent which increases expression or activity of a Hsp40 chaperone.

2. The method of claim 1 further comprising administering to the subject a Hsp70 chaperone or an agent which increases expression or activity of a Hsp70 chaperone.

3. The method of claim 1 wherein the Hsp40 chaperone is selected from the Hdj1 class of chaperones.

4. A method for suppressing polyglutamine toxicity in a subject comprising administering to the subject a Hsp40 chaperone or an agent which increases expression or activity of a Hsp40 chaperone.

5. The method of claim 4 further comprising administering to the subject a Hsp70 chaperone or an agent which increases expression or activity of a Hsp70 chaperone.

6. The method of claim 4 wherein the Hsp40 chaperone is selected from the Hdj1 class of chaperones.

7. A method for treating a subject suffering from a neurodegenerative disorder associated with abnormal protein aggregation comprising administering to the subject a Hsp40 chaperone or an agent which increases expression or activity of a Hsp40 chaperone.

8. The method of claim 7 further comprising administering to the subject a Hsp70 chaperone or an agent which increases expression or activity of a Hsp70 chaperone.

9. The method of claim 7 wherein the Hsp40 chaperone is selected from the Hdj1 class of chaperones.

10. A method for identifying an agent useful in treating neurodegenerative disorders associated with abnormal protein aggregation comprising determining a test agent's ability to increase expression or activity of a Hsp40 or Hsp70 chaperone wherein the ability of the test agent to increase expression or activity of the Hsp40 or Hsp70 chaperone is indicative of the test agent being useful in treating neurodegenerative disorders associated with abnormal protein aggregation.

11. The method of claim 10 wherein the Hsp40 chaperone is selected from the Hdj1 class of chaperones.

12. A composition for treatment of a neurodegenerative disorder associated with abnormal protein aggregation comprising:
(a) a Hsp40 chaperone or an agent which increases activity or expression of a Hsp40 chaperone; and
(b) a Hsp70 chaperone or an agent which increases activity or expression of a Hsp70 chaperone.

13. The composition of claim 12 wherein the Hsp40 chaperone is selected from the Hdj1 class of chaperones.

14. The composition of claim 12 wherein at least one of the agents augments levels of Hsp40 and Hsp70 induced as part of a stress response in the subject.


SEQUENCE LISTING (6 sequences)

SEQ ID NO: 1 - DNA, 45 bp, Artificial sequence
  Synthetic
cggaattcgtcgacatgggcaaagacttctacaagattctgggcc

SEQ ID NO: 2 - DNA, 69 bp, Artificial sequence
  Synthetic
ggtctagactacttgtcgtcgtcgtcatccttgtaatcgttgggcagcagctccgacagctgattctgc

SEQ ID NO: 3 - DNA, 52 bp, Artificial sequence
  Synthetic
cgctcgaggtcgacatggacaacctaaatttatacgacgttcttaaagtggc

SEQ ID NO: 4 - DNA, 73 bp, Artificial sequence
  Synthetic
ggtctagactacttgtcgtcgtcgtcatccttgtaatcagccgtctggcactgtacgccctcaaagtgagacg

SEQ ID NO: 5 - DNA, 41 bp, Artificial sequence
  Synthetic
cggaattcgtcgacatgggaccagatggcggcggtcagccg

SEQ ID NO: 6 - DNA, 29 bp, Artificial sequence
  Synthetic
ggatcaacggactagatctgccggtgccc